(19) United States
(12) Patent Application Publication
(10) Pub. No.: US 2008/0026038 A1
(43) Pub. Date: Jan. 31, 2008

(54) NO LAXATION, LOW FLATULENCE BULKING SYSTEM

(75) Inventors: George E. Steele, Fort Worth, TX (US); Donna Henry, Paris, TX (US)

Correspondence Address: FULBRIGHT & JAWORSKI L.L.P, 2200 ROSS AVENUE, SUITE 2800, DALLAS, TX, 75201-2784, US

(73) Assignee: Remington Direct LP, Fort Worth, TX (US)

(21) Appl. No.: 11/828,915

(22) Filed: Jul. 26, 2007

Related U.S. Application Data
(60) Provisional application No. 60/833,551, filed on Jul. 26, 2006.

(51) Int. Cl. A23L 1/05; A21D 10/00; A23L 1/22; A61K 9/68

(52) U.S. Cl. 424/440; 426/533; 426/576; 426/578

(57) ABSTRACT

A no-laxation, low flatulence bulking system and a method for preparation of same is disclosed. Gelatin, pectins and gellan gum are combined to form a dry-blended mixture. A second set of ingredients comprising at least erithritol is combined. A first liquid mixture comprising modified polydextrose and water is formed. The first dry-blended mixture is added to the first liquid mixture, forming a first mix. The second dry-blended mixture is added to the first mix, dimethicone is added, and the combination is heated to a first predetermined temperature. The heat is then reduced to a second predetermined temperature, and a second liquid mixture comprised of at least one acid and water is added. The second liquid mixture also may preferably include fruit concentrate, flavor, color and/or sucralose. The resultant solution is then preferably stirred constantly and held in a kettle or mixing tank for panning or molding as desired.

### CROSS-REFERENCE TO RELATED APPLICATIONS

The present application claims priority to co-pending U.S. Provisional Patent Application No. 60/833,551 entitled “No Laxation Bulking System,” filed Jul. 26, 2006, the disclosure of which is hereby incorporated herein by reference.

### TECHNICAL FIELD

The present invention relates generally to no laxation, low flatulence bulking systems and more particularly, to a system and method of forming a no laxation, low flatulence bulking system in the form of a soft candy product for human ingestion.

### BACKGROUND OF THE INVENTION

In recent years, controlling sugar, calories, fat and carbohydrates in the human diet has become more of a concern. Many humans desire to control these items to satisfy special dietary needs, i.e., individuals suffering from diabetes or other health problems. Other humans are attempting to eat more healthfully as a preventative measure and/or so as to address weight loss or maintenance concerns.

Regardless what type of dietary needs a person has, typically snack foods are part of such diet. For example, those attempting to lose weight may ingest a number of small meals during the day, some of those meals being snack foods. Similarly, diabetics may require snack foods during the day in order to maintain a proper blood sugar level. Snack foods also may be eaten in order to provide energy or to deliver vitamins to the body. Thus, most humans incorporate snack foods into their diet, but the desire is to have a more healthful snack food. To prevent hunger, there exists a need for a food item to act as a low calorie stomach fill; this food item should be able to be consumed in large quantities to satiate hunger with no resultant weight gain.

Many consider confectionery items, such as candies, to be snack foods. In the past, candies have not been considered to be very healthy. In recent years, however, many confectioners begun developing ways to make more healthful confectionery items, such as sugar-free candies. The confectionery industry has been developed around the properties of one ingredient—sucrose—and thus. when developing sugar-free candies, the desire is to find substitutes for sucrose that most closely mirror the properties, particularly the taste, of sucrose.

Polyols are often used as substitutes for sucrose in the manufacture of sugar-free confectionery items. Polyols typically used in sugar-free confectionery items include sorbitol, maltitol, lactitol, isomalt and polydextrose. Maltitol, which is produced by hydrogenating maltose in the form of a pure maltose glucose syrup, is believed to most closely resemble the properties of sucrose in that, like sucrose, maltitol is a disaccharide. However, the use of polyols, particularly maltitol, in these sugar-free confectionery items can often be disadvantageous due to the laxative and flatulence-related effects that these polyols produce. The laxative and flatulence-related effects are osmotic in origin given that the unabsorbed material upsets the osmotic balance within the intestinal system, and the consequences can be unpleasant for many who ingest sugar-free confectionery items containing such polyols. Thus, there exists a need for a bulking system, such as in a soft candy product, that is sugar-free while avoiding the laxation and flatulence-related effects typically encountered with food items that incorporate polyols.

Sugar-free confectionery products are typically manufactured in a manner similar to that of sugar-containing confectionery products. In the case of soft candy products, the amount of time needed for the products to set prior to packaging and distribution is significant given that the formulation takes a long time to dry and cool. For example, when a confectionery item containing maltitol syrup is formed, typically it takes at least one to two days before the confectionery item has sufficiently dried and cooled. Thus, there exists a need for a method of forming a bulking system that sets, dries and cools in a shorter period of time. There also exists a need for a method of packaging a bulking system in a manner that maintains the form and quality of the bulking system.

### BRIEF SUMMARY OF THE INVENTION

The present invention is directed to a method for producing a no-laxation, low flatulence bulking system. A first set of ingredients comprising gelatin, pectins and gellan gum is blended. The next step includes blending a second set of ingredients comprising at least erithritol. A first liquid mixture of water and modified polydextrose is then blended. The first set of ingredients is added to first liquid mixture, forming a first mix. Dimethicone is then added to the first mix. The second set of ingredients is then added to the combination, and the combination is heated to a first predetermined temperature. The first predetermined temperature is maintained for a predetermined time period. Then the heat is reduced to a second predetermined temperature. Once the second predetermined temperature range is reached, a second liquid mixture comprised of at least one acid and water is added to the solution. The resultant solution is then preferably stirred constantly and held in a kettle or mixing tank for panning or molding as desired. Embodiments of the invention further comprise adding a food-grade plasticizing agent, hydrogenated starch hydrolysate (HSH) and/or active ingredients as components of the no-laxation, low flatulence bulking system. Other additives also may preferably include flavors, colors, fruit concentrates and/or sucralose or another high intensity sweetener. The second set of ingredients also may comprise beta cyclodextrin and propylene glycol alginate.

Another embodiment of the invention is a no-laxation, low flatulence bulking system. The bulking system is preferably comprised of a dry-blended mixture comprising type A gelatin, pectins and gellan gum, a second dry-blended mixture comprising erithritol, a first liquid mixture of modified polydextrose and water, and a second liquid mixture comprising at least one acid and water. The first dry-blended mixture is added to the first liquid mixture to form a first mix. The second dry-blended mixture is added to the first mix and heated. The solution is reduced to a predetermined temperature, and a second liquid mixture comprised of at least one acid and water is added. The resultant solution is then preferably stirred constantly and held in a kettle or mixing tank for panning or molding as desired. A food-grade plasticizing agent also is preferably included as part of the bulking- system.

Further embodiments of the present invention are no-laxation, low flatulence gummy candy products. The gummy candy product preferably is comprised of a first dry-blended mixture, wherein the first dry-blended mixture comprises 3 to 13% type A gelatin, 0 to 5% low methoxal pectin, 0 to 5% high methoxal pectin, and 0 to 5% gellan gum. The gummy candy product is further comprised of a second dry-blended mixture comprising 10 to 50% erithritol, and in some embodiments, 0 to 6% propylene glycol alginate and 0 to 2% beta cyclodextrin. A first liquid mixture comprising 0 to 20% modified polydextrose and water and a second liquid mixture comprising 0 to 5% acid and water also form part of the no-laxation, low flatulence gummy candy product contemplated by the present invention. The first dry-blended mixture is added to the first liquid mixture to for a first mix. The second dry-blended mixture is then added to the first mix along with a small amount of dimethicone and heated. A food-grade plasticizing agent is preferably added to the combination. The heat is then reduced, and the second liquid mixture is added. The resultant solution is then preferably stirred constantly and held in a kettle or mixing tank for panning or molding as desired.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

The desire is to achieve a stable gelatin matrix using a bulking agent. This is achieved by using a sugar alcohol, such as erithritol, as well as a modified polydetrose, such as Litesse Ultra liquid, having high fiber content. The finished product includes high levels of erithritol dissolved in water and stabilized by hydrocolloids. The finished product is preferably zero sugar and low calorie providing for no taxation and low flatulence properties upon ingestion. A no taxation, low flatulence bulking system, such as a gummy candy product, is preferably formed using a dry-blended mixture of pectins, type A gelatin and gellan gum added to a first liquid mixture of water and modified polydextrose to form a first mix. Dimethicone, an ingredient having anti-gas properties, is then added to the first mix. A second set of ingredients including erithritol, beta cyclodextrin and propylene glycol alginate are mixed together, added to the combination, and preferably the combination is stirred constantly until any lumps that may be present in the combination are dispersed. Heat is then applied to the solution until a first predetermined temperature level is reached. Preferably, this predetermined temperature is approximately 160 degrees Fahrenheit, although the predetermined temperature may vary depending on the scaling of the manufacturing process. The temperature is maintained at a first predetermined level for a specified amount of time, and then the heat is reduced to a second predetermined temperature. This second predetermined temperature is preferably a low temperature, such as 100 to 110 degrees Fahrenheit, although again the temperature may vary depending on the scaling of the manufacturing process. A food-grade plasticizing agent is preferably included as part of the solution. Once the solution has reached the second predetermined temperature ranges a second liquid mixture comprised of at least one acid and water is added to the solution. The resultant solution is then preferably stirred constantly and held in the kettle or mixing tank for panning or molding as desired. The product also may be extruded. Plating or molding is then conducted into sprayed or prepared pans. The pans are allowed to cool, preferably for one to two hours, and then the resulting product is depanned.

Gelatins are often used for making confectionery items. Gelatins produced by acid hydrolysis are referred to as type A, whereas gelatins produced by alkaline hydrolysis are typically referred to as type B. The pH of type A gelatin is typically higher than the pH of type B gelatin (6.3-9.5 versus 4.5-5.2). Type A gelatins are preferably used for forming no-laxation, low flatulence bulking systems contemplated by the present invention. Gelatins are typically not stable in acid, and thus any addition of acid should be as late as possible in the manufacturing process. Specifically, a gelatin type A 250 bloom is preferred. This gelatin has been selected based on having the specific ability to allow gelatin to set at a lower temperature (i.e., reduce the amount of time for setting as well as the temperature at which setting occurs) when used in combination with other ingredients as described and to produce a firm soft or hard candy product. Further, type A gelatin has the ability to work in conjunction with low methoxyl pectin and gellan gums.

Pectins also are often used for making confectionery items. However, typically they have not been used in conjunction with gelatins. This is due to the problem that if the pH does not fall within a specific range, the system formed may be unstable and may not set properly. However, in the present invention pectin amid AF020 is a preferable pectin because of its specific ability to work with type A gelatin in setting the bulking system without resulting in stickiness of the product when placed in combination with sugar alcohols or polyols. A low methoxyl pectin, such as pectin LM/CP Kelco, is also used and is chosen for its ability to work with type A gelatin and gellan gums in setting a firm gummy candy product.

Gellan gums are preferably used in forming the bulking system contemplated by the present invention so as to give the resultant gummy candy product, for example, its elastic texture. A preferable gellan gum is Kelcogel LT100 gellan gum as it is a texture modifier and stabilizer, and provides for an elastic gel texture when used in food-related applications. Beta cyclodextrin is included as part of the second dry-blended mixture for its ability to bind water in conjunction with gellan gums to form a firm gel.

In the first step of the bulking system manufacturing process, type A gelatin, pectins (pectin amid AF020 and pectin LM) and gellan gum are dry-blended to form a first dry-blended mixture. The mixture is typically combined through blending using a mixer run at a slow speed for a specified period of time until even dispersement is achieved. Approximately two minutes is needed to form the dry-blended mixture such that the gelatin, pectins and gellan gum are evenly dispersed. No heat should be applied to the gelatin/pectin combination. The bulking system is typically comprised of 3 to 13% type A gelatin, 0 to 5% low methoxyl pectin, 0 to 5% high methoxyl pectin, and 0 to 5% gellan gum. Preferably, however, the gelatin comprises approximately 7 to 8% of the bulking system, and the pectins comprise approximately 1% of the resultant bulking system. However, as the amounts and percentages of gelatin, pectins and/or gellan gum change due to scaling of the manufacturing process, it should be appreciated that the time required to fully blend these components also may change.

In the second step of the bulking system manufacturing process, erithritol, beta cyclodextrin and propylene glycol alginate are combined to form a second dry-blended mixture. Erithritol is a polyol that acts as a novel bulk sweetener and has a caloric value close to zero. It is the only polyol presently known that has been shown not to cause laxation effects when incorporated into sugar-free edible items. Erithritol is believed to avoid this laxative effect because it is excreted through the kidneys, and as it has a low molecular weight, more than 90% is absorbed in the small intestine. This fraction is not metabolized and is excreted unchanged through the urine. Thus. it is shown to have the highest digestive tolerance of the polyols as studies have shown that adults investing up to 1 g of erithritol per day per kilogram of body weight do not show any gastrointestinal effects. Erithritol granules are preferably used in the bulking system contemplated by the present invention. Granules, as opposed to a powder form, are used as the powder version typically does not retain enough water for absorption. This lack of water retention may affect the process to the extent that setting of the bulking system may be hindered or even prevented.

Like the first dry-blended mixture, this second dry-blended mixture is typically formed using a blender or mixer run at a slow speed for a specified period of time. Approximately two minutes is preferably needed to form the second dry-blended mixture such that the components are evenly dispersed. No heat should be applied to this second dry-blended mixture. The bulking system is typically comprised of 0 to 6% propylene glycol alginate, 0 to 2% beta cyclodextrin and 10 to 50% erithritol. Preferably, however, propylene glycol alginate comprises approximately 0 to 1% of the bulking system, beta cyclodextrin comprises approximately 0 to 2% of the bulking system, and erithritol comprises approximately 20 to 30% of the bulking system. However, as the amounts and percentages of the components change due to scaling of the manufacturing process it should be appreciated that the time required to fully blend the components also may change.

A food-grade plasticizing agent is preferably incorporated into the bulking system. Several types of food-grade plasticizing agents may be used. In one embodiment of the present invention, in an intermediate step between the first and second steps of the bulking system manufacturing process, a plasticizing agent, such as paraffin wax or carnuba wax may be weighed and melted in a kettle or other metal container. Preferably, the wax will melt at approximately 200-212 degrees Fahrenheit. Paraffin wax if used preferably comprises approximately 0 to 20% of the resultant bulking system. If carnuba wax is used, it preferably comprises 0 to 60% of the resultant bulking system. This melted wax should be held for later addition in the bulking manufacturing process. In another embodiment of the present invention, a food-grade resin such as, but not limited to, shellac resin, may be incorporated in the second dry-blended mixture or alternatively this food-grade resin may be added in a separate step prior to addition of the second liquid mixture to the solution as will be later described, and this food-grade resin preferably comprises 0 to 20% of the resultant bulking system. In a further embodiment of the present invention, a food-grade gum, such as, but not limited to, mastic gum and pullulan gum, may be incorporated into the second dry-blended mixture, and this food-grade gum preferably comprises 0 to 20% of the resultant bulking system.

In a third step of the bulking system manufacturing process, water and modified polydextrose are combined to form a first liquid mixture. A preferred modified polydextrose for use is Litesse. Litesse is known as a specialty carbohydrate (polydextrose) that replaces sugar and fat while improving flavor, texture and mouthfeel in a variety of applications. It is low glycemic and thus suitable for consumers seeking low impact carbohydrates. Further, Litesse is water soluble and is used as a bulking agent to make a variety of items lower in fat, calories and sugar-free while also high in fiber and having a good taste. The liquid formulation of Litesse (Litesse Ultra liquid) is preferable to use because there is typically less water present in the formulation, and it presents a clean taste with mildly sweet flavor. The percentage of Litesse Ultra liquid preferably present in the formulation can rankle from 0 to 20% of the resultant bulking system. This low percentage range for Litesse Ultra liquid is achieved by incorporating it with type A gelatin in conjunction with gellan gum and low methoxyl/high methoxyl pectins to produce a firm gummy. It should be appreciated however that lower percentages of Litesse Ultra liquid may be used if another form of Litesse (such as Litesse II) is also used in the composition and/or a hydrogenated starch hydrolysate (HSH) also is employed. It also should be appreciated that a modified polydextrose other than Litesse may be employed without departing from the objects of the present invention.

Water and modified polydextrose are preferably combined in a mixing kettle or tank. This mixing process may occur by metering water into the mixing kettle or tank or by weighing the water in a separate container and then adding to the modified polydextrose. This first liquid mixture should not be heated.

An intermediate step in the bulking system manufacturing process may preferably take place between the second and third steps described above. This intermediate step involves preparing a mixture of potassium sorbate and sodium hexametaphosphate (Glass H) for addition to water in the mixing kettle or tank before the first dry-blended mixture and first liquid mixture are combined in the tank. Sodium hexametaphosphate and potassium sorbate are preservatives used to keep the bulking system fresh and to prevent mold from forming in the product.

If hydrogenated starch hydrolysate (HSH) is to be included as part of the bulking system, it should be prepared for use following completion of the third step of the bulking manufacturing process. A hydrogenated starch hydrolysate, such as HSH Stabilite SD30 may preferably be used as a bulking agent. SD30 is a hydrogenated starch hydrolysate in spray-dried form that is a low-sweetness powder and can be dissolved in water to produce clear, noncrystallizing syrups. When it is combined with water, the water must be maintained at room temperature in order to ensure adequate mixing and dispersement of the ingredient in solution. The HSH should be weighed and held in a separate container and then mixed into the second dry-blended mixture at this stage in the manufacturing process. Should HSH be included as part of the bulking system, it preferably comprises 0 to 4% of the resultant bulking system.

If an active ingredient is to be included as part of the bulking system, it should be weighed and held in a separate container. Active ingredients include, but are not limited to: vitamins; minerals; mineral salts; caffeine; pheobromine; central nervous system stimulants; amino acids; appetite suppressants; SSRIs; MAOI's; electrolytes; hydroxy citric acid; 5-hydroxy tryptophan (5-HTP); NSAids including acetaminophen, ibuprophen, aspirin or salicylic acid; glycerol; weight loss ingredients; and over-the-counter (OTC) medicines including, but not limited to, allergy/sinus medicines (such as diphenhydramine HCl), cough suppressants (such as dextromephorphan HBr), antihistamines, and nasal decongestants (such as pseudoephedrine HCl) may be added following addition of HSH, if desirable. These active ingredients, if incorporated into the hulking system, typically comprise approximately 3-4% of the resultant bulking system but may range from 0 to 7%. This system for delivery of active ingredients is useful for children as well as adults who would express a preference for ingesting these active ingredients in a soft candy form which may have a more pleasant taste and may be more enjoyable to consume than an active ingredient in pill form, for example.

Dimethicone is also known as polydimethylsiloxane (PDMS) and is recgonized for its unusual rheological properties. Similar to the weighing of active ingredients, dimethicone or other ingredients having anti-gas properties should be weighed and held in a separate container for later use in the manufacturing process.

A second liquid mixture is formed by mixing together at least one acid and a small amount of water together in a separate container and blending until the combination is mixed and the color is well dispersed. Different acids may be utilized depending on the type of finished product desired. Malic acid is preferably used when a fruit-flavored product is formulated. Malic acid has characteristics that allow for setting the bulking system at a low temperature of approximately 155-160 degrees Fahrenheit, Ascorbic acid and sodium acid sulfate (also known as pHase) also are preferably used along with fumaric acid to set the gel formed as well as to retard flatulence. It should be appreciated that the amount of acid used in the second liquid mixture may comprise anywhere from 0 to 5% of the resultant bulking system. Preferably, the acids comprise 1 to 2% of the bulking system and are utilized, in part, to activate the gels in the bulking system to set the formation into a gummy candy product. Although only a combination of at least one acid and water is required to form the second liquid mixture, it should be appreciated that at this step, color, flavors, fruit concentrate and sucralose sweetener or some other high intensity sweetener may be blended with at least one acid and water to form the second liquid mixture.

Preferred flavors may be fruit flavors, although other flavors may be added without departing from the object of the invention. However, whatever flavors utilized in forming the second liquid mixture are preferably concentrated and are in liquid form the majority of the time. Preferably the flavor level is at 2% or below. It should be appreciated that when concentrations of the flavor fall below this percentage, many off flavors begin to show up in the finished bulking system due to the carriers in the flavors and the reduction of the other ingredients to make a place for the carriers for flavors.

For added flavor in fruit-flavored bulking systems. a fruit concentrate with essence returned is preferably utilized. For example, strawberry fruit puree concentrate with essence returned is a preferable fruit concentrate as it is low in calories and is 28 degree brix. It also has the essence returned allowing more flavor for a smaller amount of usage. This fruit concentrate typically comprises less than 1% of the resultant bulking system although it can range from 0 to 5% of the composition. In a preferred embodiment, a pear puree concentrate with essence returned is utilized to allow other flavor profiles to be incorporated into the resultant bulking system. A fruit concentrate is preferably utilized in formation of a bulking system contemplated by the present invention for its functional attributes of adding particle fibrous pieces and pectin to the bulking system and also because of its high refractive index.

Dried sucralose sweetener is preferably used for additional flavoring and comprises 0 to 5% of the bulking system, although preferably sucralose comprises less than 1% of the bulking system. A dried form of sucralose is preferred in that the liquid form has polyesters/polysorbates that may adversely affect the resulting bulking system. Various colors can be used depending on the color desired for the bulking system, and the color typically comprises less than 0.01% of the bulking system, When at least one acid, color, flavors, sucralose, water and fruit concentrate are combined to preferably form the second liquid mixture, the components are combined until mixed and the color is well dispersed. The second liquid mixture is then set aside.

Once the second liquid mixture is formed, the preservatives should be added to water in the mixing tank or kettle and mixed until dissolved. Alternatively, the preservatives should be added to the water and then allowed to sit while other ingredients are being weighed so that the preservatives are given time to dissolve prior to the addition of other ingredients.

After the various dry-blended and liquid mixtures have been prepared, the first dry-blended mixture should be added to the first liquid mixture which is already present in the mixing tank or vessel. This combination should be mixed quickly and briskly, preferably using a lightning mixer or an equivalent mixer at a slow to moderate speed until the solid portions of the mixture have been dispersed but prior to the mixture starting to thicken. No heat should be added to this combination. Dimethicone should then be added to the mixing tank under continuous agitation and stirred at a slow to moderate speed to disperse the solid portions of the mixture without thickening the mixture. Again, no heat should be added at this step.

The second dry-blended mixture should then be added to the combination of ingredients already present in the mixing tank. This combination should be mixed quickly and briskly with a lightning mixer or other equivalent mixer at medium speed until the solid portions of the mixture have been dispersed. Preferably, the dispersion of the solid portions of the mixture should occur after approximately 15 minutes of continued agitation. Heat preferably should be applied to the mixing tank or vessel while this mixture is being blended; however, heat may be applied prior to blending the mixture depending on the amount of time required for the mixer or tank to reach optimal temperature. It should be appreciated that the mixture must be fully dispersed before the heat is applied to the mixtures otherwise, solid lumps may appear in the mixture. It also should be appreciated that the mixture will be fairly thick prior to the application of heat, but the mixture will transform Into a liquid and thicken upon reaching a first predetermined temperature of approximately 160 degrees Fahrenheit.

Once this first predetermined temperature has been reached, it should be maintained for a period of time dependent on the type of equipment and amounts of ingredients used but preferably for approximately two minutes. While holding at the first predetermined temperature, the mixture is preferably stirred or agitated constantly. At this step, should a plasticizing agent such as melted paraffin wax or carnuba wax be incorporated into the bulking system, it should preferably be added to the combination under constant slow to medium agitation. However, if other food-grade plasticizing agents such as food-grade resins or food-grade gums are to be utilized, these plasticizing agents are added as part of the second dry-blended mixture or in the alternative separately prior to addition of the second liquid mixture. After maintaining the first predetermined temperature for a period of time and, if a wax plasticizing agent is used, once the combination of ingredients including the melted max has fully blended, the heat being applied to the mixture is preferably reduced to a low temperature in the range of 100 to 110 degrees Fahrenheit. This second predetermined temperature of approximately 100 degrees Fahrenheit is a temperature that is preferable for filling trays to perform the molding process. If an active ingredient is to be utilized, it should be added to the mixture while stirring constantly until the solid portions of the solution are dispersed. This active ingredient should be added while no heat is being applied to the mixing tank.

As the mixture is cooling in the mixing tank, the second liquid mixture should be added to the mixing tank under continuous agitation at medium speed. Once this mixture has been added, the resulting, composition may be plated or molded in sprayed or prepared pans after the color has been dispersed. It should be appreciated that the bulking system should be plated or molded as quickly as possible as it will typically begin to Gel upon cooling. Once the composition has been plated or molded, it should be allowed to cool before depanning should be performed. Alternatively, the resultant composition may be extruded and allowed to cool.

Upon cooling and depanning or extruding, the bulking system is preferably packaged in a container, such as a sealable bag. and a nitrogen drop or flush is preferably introduced into the container. This nitrogen introduction is beneficial to maintaining the form and quality of the resultant product in that the nitrogen preferably stops an oxidation process from taking place, allowing the resultant bulking system to retain the flavors and colors in the form that they were introduced into the bulking system. Further, as erithritol comprises a relatively large percentage of the resultant product, introduction of nitrogen preferably retards its crystallization as embodiments of the bulking system which are devoid of nitrogen may only be comprised of as little as one-fourth the percentage of erithritol found in the preferred embodiment of the present invention. In additions nitrogen preferably acts as a thermal insulator to slow down any melting that the resultant product may undergo given that typically sugar-free bulking, systems have had a tendency to melt at lower temperatures than are desirable, but introduction of nitrogen into the sealable container holding the bulking system preferably counteracts such tendency.

The process described above used to make the novel bulking system is devised so as to produce a product that will plate, mold and set faster than prior soft or hard candy products. Temperatures used when the product is at the stage for heating and molding should be kept to a maximum of 165 degrees Fahrenheit. Preferably, the soft or hard candy product should not remain in the mixer for more than two hours prior to heating or molding. Once the heating steps are complete, the product should be molded immediately and not left to stand in the mixer or in packaging equipment. Further, the components of the bulking system should not be left in a pre-measured or pre-weighed state for more than approximately two hours because of the hydroscopic nature of the ingredients.

It is preferable that the setting temperature be maintained at approximately 160 degrees Fahrenheit. This setting temperature is important for loading active ingredients such as vitamins, over-the-counter (OTC) drugs, as well as acetaminiophens (or NSAids) because high heat exposure potentially will destroy these active ingredients. The setting temperatures are quite cool in that the finished product gels and sets below 180 degrees Fahrenheit which is below the melting point of every NSAid currently on the market.

Finished products can include weight loss snacks and bars that can be either extruded or molded. When a reference is made to extrusion, this is usually when the product is squirted out and then cut, such as when licorice rope or bars are made. When molded, such as molding that takes place with gummy bears, the molds are pressed into cornstarch, the melted ingredients are poured in, and then are finished with a carnouba wax in a panning process. Weight loss/energy/meal replacement bars are extruded. In order to make a bulking system extrudable, one adds flour or a flour analog. Preferably, the flour or flour analog comprises 0 to 15% of the resultant bulking system. A type of flour that may be used is Konjac flour which has a high fiber content (such as 95%). The use of rice flour or starch are other possibilities. The preference is to use a flour that has no glutens. A soy milk powder also may be preferably included with the flour or flour analog to form an extrudable finished product, and if soy milk powder is used, it also preferably comprises 0 to 15% of the resultant bulking system.

Different types of finished product applications formed by the method described in the present invention include low-caloric gummy products, non-rolled fruit snacks, gummy products loaded with vitamins, energy-producing gummy products, gummy products for weight loss, chocolate chews, fruit extruded bar or rope/twist. as well as hard candies, bars, licorice ropes or analogs, fruit snacks, and rolled fruit snacks. Rolled fruit snacks are made by spraying a thin layer of mixed, molten liquid onto wax paper or some other paperlike substrate. Rolled fruit snacks or other fruit snack products, in the context of the described bulking system invention contain additionally a low dextrose equivalent (low DE) fruit concentrate with returned fruit essence. While these types of bulking systems are specifically identified, it should be appreciated that other bulking systems may be produced by the process discussed in the context of this invention.

In one embodiment of the present invention, a gummy candy product having a fruit flavor and loaded with dimethicone is formed. Gelatin type A 250 bloom, pectin amid AF020, pectin LM/CP Kelco and gellan gum are combined to form a first dry-blended mixture. Gelatin type A preferably comprises approximately 8% of the resultant bulking system, pectins comprise approximately 1-2% of the resultant composition and gellan gum comprises approximately 0 to 1% of the resultant composition. Erithritol, beta cyclodextrin and propylene glycol alginate are combined to form a second dry-blended mixture. Erithritol granules preferably comprise approximately 20-30% of the resultant composition while beta cyclodextrin comprises approximately 0-2% of the composition and propylene glycol alginate comprises approximately 0 to 5% of the resultant bulking system. A first liquid mixture is preferably comprised of water and Litesse Ultra liquid. Litesse Ultra liquid comprises approximately 8-10% of the resultant bulking system. A second liquid mixture is prepared comprising ascorbic acid, malic acid, fumaric acid, sodium acid sulfate, fruit concentrate, colors, flavor and sucralose. Acids comprise approximately 2-3% of the resultant bulking system, and the remaining components of the second liquid mixture comprise approximately 1-2% of the resultant composition. Preservatives (such as Glass H and potassium sorbate) are added to water in the mixing tank and then dissolved.

The first liquid mixture should then be added to the mixing tank and then a first dry-blended mixture is combined with the liquid mixture under agitation. Dimethicone is then added to the liquid mixture followed by the second dry-blended mixture. Following combination of the above-described ingredients (with the exception of the second liquid mixture), heat is applied to the solution to reach a first predetermined temperature of preferably 160 degrees Fahrenheit. Preferably a food-grade plasticizing agent is added to the solution, either in a separate step or as a part of the second dry-blended mixture. The food-grade plasticizing agent preferably comprises 0 to 20% of the resultant bulking system: however, if carnuba wax is selected as the food-grade plasticizing agent, it preferably comprises 0 to 60% of the resultant bulking system. The solution is held at this first predetermined temperature for a specified period of time, and then the heat is preferably reduced to a second predetermined temperature range of approximately 100 to 110 degrees Fahrenheit. When the second predetermined temperature range is reached, the second liquid mixture is added to the solution, and the composition. that forms is held for later panning or molding.

Variations of the above described embodiment may be formed wherein, for example, by addition of active ingredients or HSH. The dry-blended mixture as well as the first and second liquid mixtures remain at the same percentages of the resultant soft candy composition even with addition of the active ingredient(s) or HSH.

While many of the embodiments described include malic acid as part of the second liquid mixture, it should be appreciated that lactic acid may be substituted for malic acid without appreciably changing the percent composition of the resultant bulking system. Lactic acid is preferably used when the bulking system is to have a brown flavor, meaning that the flavor has been typically derived from two basic thermal processes: caramelization and Maillard reactions. Brown flavors include, but are not limited to, chocolate. vanilla, toffee, mocha, cream/milk, cinnamon and caramel. If lactic acid is preferably used as part of the formulation of the bulking system, it should be appreciated that the colors and flavors incorporated into the second liquid mixture will be altered as fruit concentrate typically would not be utilized when a brown flavor is desired.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly. the appended claims are intended to include within their scope such processes, machines, manufacture, compositions of matter, means. methods, or steps.

What is claimed is:

1. A low-flatulence, no-laxation gummy candy product, said gummy candy product comprising:
a first dry-blended mixture, said first dry-blended mixture comprising 3 to 13% type A gelatin, 0 to 5% low methoxal pectin, 0 to 5% high methoxal pectin, and 0 to 5% gellan gum;
a second dry-blended mixture, said second dry-blended mixture comprising 10 to 50% erithritol;
a first liquid mixture, said first liquid mixture comprising 0 to 20% modified polydextrose and water; and
a second liquid mixture, said second liquid mixture comprising 0 to 5% acid and water;
wherein said first dry-blended mixture, said second dry-blended mixture, said first liquid mixture, said second liquid mixture and a food-grade plasticizing agent are combined.

2. The gummy candy product of claim 1, said second liquid mixture further comprising:
color, flavor, fruit puree concentrate with essence returned, and sucralose.

3 The gummy candy product of claim 2, said gummy candy product further comprising:
0 to 3% flavor,
0 to 5% sucralose: and
0 to 15% fruit puree concentrate with essence returned.

4. The gummy candy product of claim 1, said gummy candy product further comprising:
an anti-gas ingredient.

5. The gummy candy product of claim 1, said gummy candy product further comprising:
hydrogenated starch hydrolysate (HSH).

6. The gummy candy product of claim 5 wherein said HSH comprises 0 to 4% of said gummy candy product.

7. The gummy candy product of claim 1, said gummy candy product further comprising:
at least one active ingredient.

8. The gummy candy product of claim 7 wherein said at least one active ingredient comprises 0 to 7% of said gummy candy product.

9. The gummy candy product of claim 1, said gummy candy product further comprising:
at least one preservative.

10. The gummy candy product of claim 9 wherein said at least one preservative is selected from the group comprising:
potassium sorbate and sodium hexametaphosphate (Glass H).

11. The gummy candy product of claim 9, wherein said at least one preservative comprises 0 to 5% of said gummy candy product.

12. The gummy candy product of claim 1, wherein said food-grade plasticizing agent is selected from the group comprising:
paraffin wax, carnuba wax, food-grade resins, and food-grade gums.

13. The gummy candy product of claim 1, said acid of gummy candy product comprising:
malic acid, ascorbic acid, fumaric acid and sodium acid sulfate.

14. The gummy candy product of claim 1, said second dry-blended mixture further comprising:
0 to 6% propylene glycol; and
0 to 5% beta cyclodextrin.

15 A method for producing a low-flatulence, no-laxation bulking system, said method comprising:
blending a first set of ingredients comprising gelatin, pectins and gellan gum to form a first dry-blended mixture;
blending water and modified polydextrose to form a first liquid mixture;
blending erithritol to form a second dry-blended mixture;
adding said first dry-blended mixture to said first liquid mixture, forming a first mix;
adding said second dry-blended mixture to said first mix to form a second mix and heating to a predetermined temperature;
adding a food-grade plasticizing agent;
maintaining said first predetermined temperature for a predetermined time period;
reducing the heat to a second predetermined temperature and holding the heat at said second predetermined temperature; and
adding a second liquid mixture comprising at least one acid and water to said second mix under agitation to form a moldable mix.

16. The method of claim 15 wherein mixing occurs at a slow to medium speed and no heating occurs when said first dry-blended mixture is added to said first liquid mixture.

17. The method of claim 15 wherein said heating to said first predetermined temperature occurs under constant agitation.

18. The method of claim 15, said method further comprising:
depanning said moldable mix into a sealable container; and
introducing nitrogen into said sealable container.

19. The method of claim 15, said method further comprising:
cooling said moldable mix; and
plating said moldable mix.

20. The method of claim 15, said method further comprising:
adding at least one preservative, wherein said at least one preservative is added prior to formation of said first mix.

21. The method of claim 15, said method further comprising:
adding an anti-gas ingredient, wherein said anti-gas ingredient is added prior to the addition of said second liquid mixture.

22. The method of claim 15, said method further comprising:
adding at least one active ingredient, wherein said active ingredient is added to the second mix after the second mix has been reduced to said second predetermined temperature.

23. The method of claim 22 wherein said active ingredient is selected from the group comprising:
vitamins, minerals, mineral salts, caffeine, pheobromine, central nervous system stimulants, amino acids, appetite suppressants, SSRIs, MAOI's, electrolytes, hydroxy citric acid, 5-hydroxy tryptophan (5-HTP), NSAids, acetaminophen, ibuprophen, aspirin, salicylic acid, glycerol, weight loss ingredients, over-the-counter (OTC) medicines, allergy/sinus medicines, diphenhydramine HCl, cough suppressants, dextromephorphan HBr, antihistamines, nasal decongestants, and pseudoephedrine HCl.

24. The method of claim 15, wherein said second liquid mixture further comprises:
colors, flavors, fruit concentrate, and sucralose.

25. The method of claim 15, said method further comprising:
adding hydrogentated starch hydrolysate (HSH) to said first mix, wherein said HSH is added to said second dry-blended mixture.

26. The method of claim 15, wherein said adding a food-grade plasticizing agent comprises:
adding a food-grade gum to said second dry-blended mixture.

27. The method of claim 15, wherein said adding a food-grade plasticizing agent comprises:
adding a solution of melted paraffin wax to said second mix.

28. The method of claim 15, wherein said adding a food-grade plasticizing agent comprises:
adding a food-grade resin to said second dry-blended mixture.

29. The method of claim 15, wherein said adding a food-grade plasticizing agent comprises:
adding a food-grade resin to said second mix.

30. The method of claim 15 wherein said second dry-blended mixture further comprises:
beta cyclodextrin; and
propylene glycol alginate.
